(19) United States
(12) Patent Application Publication
(10) Pub. No.: US 2026/0218103 A1
(43) Pub. Date: Jul. 30, 2026

(54) Semiconductor Device for Culturing Cells

(71) Applicant: Imec vzw, Leuven (BE)

(72) Inventors: Dries Braeken, Waasmont (BE); Maria del Mar Condor, Leuven (BE)

(21) Appl. No.: 19/141,581

(22) PCT Filed: Dec. 20, 2023
(86) PCT No.: PCT/EP2023/087063

§ 371 Date: Jun. 20, 2025

(30) Foreign Application Priority Data
Dec. 21, 2022 (EP) 22215627.5

(51) Int. Cl. C12M 3/06; C12M 1/00; C12M 1/12; C12M 1/34

(52) U.S. Cl. CPC C12M 23/16; C12M 23/22; C12M 23/34; C12M 23/58; C12M 25/14; C12M 29/18; C12M 41/36

(57) ABSTRACT

An aspect may include a semiconductor device comprising a first unit comprising a semiconductor substrate with through holes, a cell culture chamber in the bottom surface, microfluidic channels crossing the top surface, and sensors/actuators attached to it. The second unit has a cell culture chamber above the top surface covered by a transparent layer, and an inlet/outlet for liquid. The bottom surface of the first unit is covered by a second transparent layer. Together, this device allows for sensing and controlling liquid within the chambers, and provides an optically transparent environment for cell culture.

### TECHNICAL FIELD OF THE INVENTION

The present invention relates to the field of cell culture, and more in particular to devices and methods for 3D cell culture of multiple cell types.

### BACKGROUND OF THE INVENTION

Organ-on-chips (OoCs) or Microphysiological Systems (MPS) are microfluidic devices containing multiple cell types and are used to emulate a human organ in a specific context, particularly in pre-clinical drug development. These devices typically consist of multiple microfluidic channels and dynamic flow conditions, and are constructed out of polymers using soft lithography or injection molding. The readout of cells inside these models is typically done using (fluorescent) microscopy. The integration of sensors, such as electrodes, into such multi-cellular microfluidic systems is difficult. Forming a complex cell culture environment by assembling a multiplicity of such devices into a stack is labor-intensive and prone to error, resulting in poor reproducibility and reliability. Additionally, there are only few sensor-integrated systems shown in literature, and they are typically limited in the number of electrodes/sensors available and do not enable real-time monitoring.

There is therefore a need in the art for new devices overcoming at least partially one or more of these issues.

### SUMMARY OF THE INVENTION

It is an object of the present invention to provide a semiconductor device that can function as an OoCs device.

The above objective is accomplished by a method and device according to the present invention.

In the first aspect, the present invention relates to a semiconductor device comprising: a. A first unit comprising: i. A first semiconductor substrate having through holes, a top surface and a bottom surface, ii. A first cell culture chamber formed in the bottom surface and in fluid communication with the through holes, iii. At least a first microfluidic channel crossing the top surface and in fluid communication with the first cell culture chamber, and iv. At least one sensor and/or an actuator attached to the top surface or the bottom surface, b. A second unit comprising: i. A second cell culture chamber formed above the top surface of the first semiconductor substrate and in fluid communication with the through holes, ii. A first optically transparent layer covering the second cell culture chamber, and iii. An inlet and an outlet for allowing fluid to enter and exit the second cell culture chamber, and c. A second optically transparent layer bonded with the bottom surface of the first semiconductor substrate and covering the first cell culture chamber.

In the second aspect, the present invention relates to a process for fabricating a semiconductor device comprising: a. providing a first semiconductor substrate having through holes, a top surface and a bottom surface; b. forming a first cell culture chamber in the bottom surface and in fluid communication with the through holes; c. forming at least a first microfluidic channel crossing the top surface and in fluid communication with the first cell culture chamber; d. forming at least one sensor and/or an actuator on the top surface or bottom surface; e. forming a second cell culture chamber above the top surface of the first semiconductor substrate such that the first cell culture chamber is in fluid communication with the second cell culture chamber; f. providing a first optically transparent layer; g. bonding the first optically transparent layer to the top surface of the second cell culture chamber so that it is covered by the first optically transparent layer; h. providing a second optically transparent layer; i. bonding the second optically transparent layer to the bottom surface of the first semiconductor substrate such that the first cell culture chamber is covered by the second optically transparent layer.

It is an advantage of embodiments of this invention that a single device can combine sensing and/or actuating elements attached to a semiconductor structure with cavities for microfluidic channels in that same semiconductor substrate.

It is an advantage of embodiments of this invention that cavities and microfluidic channels of accurate and small dimensions can be implemented. This is enabled by the semiconductor technology.

It is a further advantage of embodiments of this invention that the integrated sensors/actuators and microfluidic channels enable real-time monitoring of cells in a complex culture environment.

It is also an advantage of embodiments of this invention that the device allows for nutrient and oxygen supply to the culture chamber through channels.

It is a further advantage of embodiments of this invention that the device, in parts, is optically transparent, allowing for microscopy.

It is a further advantage of embodiments of this invention that the top layer of the device features all microfluidic connections to the channels.

It is a further advantage of embodiments of this invention that a reproducible, scalable, automatable, and reliable process for forming the device is provided.

Particular and preferred aspects of the invention are set out in the accompanying independent and dependent claims. Features from the dependent claims may be combined with features of the independent claims and with features of other dependent claims as appropriate and not merely as explicitly set out in the claims.

Although there has been constant improvement, change, and evolution of devices in this field, the present concepts are believed to represent substantial new and novel improvements, including departures from prior practices, resulting in the provision of more efficient, stable, and reliable devices of this nature.

The above and other characteristics, features, and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic representation of a vertical cross-section through a semiconductor device according to an embodiment of the present invention wherein a first, second, and third cell culture chambers are present.

FIG. 2 is a transparent schematic representation of a perspective view of a semiconductor device according to an embodiment of the present invention wherein only a first and a second cell culture chambers are present.

FIG. 3 is a transparent schematic representation of a perspective view of a first optically transparent layer suitable for bonding to the top surface of the second cell culture chamber of a semiconductor device according to an embodiment of the present invention.

FIG. 4 enlarges a part of FIG. 2.

FIG. 5 is a schematic representation of a vertical cross-section through a semiconductor device according to an embodiment of the present invention wherein the second optically transparent layer is an active sensing platform.

In the different figures, the same reference signs refer to the same or analogous elements.

### DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second, third, and the like in the description and in the claims, are used for distinguishing between similar elements and not necessarily for describing a sequence, either temporally, spatially, in ranking, or in any other manner. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

The terms “over” and “above” are used as synonyms and cover situations with and without physical contacts. The term “on” means “over and in physical contact with”.

It is to be noticed that the term “comprising”, used in the claims, should not be interpreted as being restricted to the means listed thereafter; it does not exclude other elements or steps. It is thus to be interpreted as specifying the presence of the stated features, integers, steps, or components as referred to, but does not preclude the presence or addition of one or more other features, integers, steps or components, or groups thereof. The term “comprising” therefore covers the situation where only the stated features are present (and can therefore always be replaced by “consisting of” in order to restrict the scope to said stated features) and the situation where these features and one or more other features are present. The word “comprising” according to the invention therefore also includes as one embodiment that no further components are present. Thus, the scope of the expression “a device comprising means A and B” should not be interpreted as being limited to devices consisting only of components A and B. It means that with respect to the present invention, the only relevant components of the device are A and B.

Similarly, it is to be noticed that the term “coupled”, also used in the claims, should not be interpreted as being restricted to direct connections only. The terms “coupled” and “connected”, along with their derivatives, may be used. It should be understood that these terms are not intended as synonyms for each other. Thus, the scope of the expression “a device A coupled to a device B” should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures, or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures, and techniques have not been shown in detail in order not to obscure an understanding of this description.

The following terms are provided solely to aid in the understanding of the invention.

When a first entity is said to be “in fluid communication” with a second entity, it is meant that at least some molecules can be exchanged between both entities.

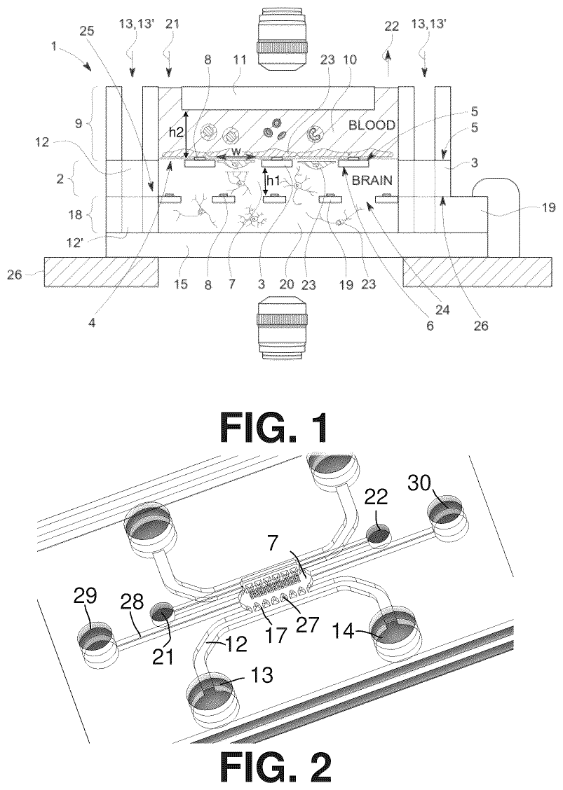

We now refer to FIG. 1. In the first aspect, the present invention relates to a semiconductor device (1) comprising: a. A first unit (2) comprising: i. A first semiconductor substrate (3) having through holes (4), a top surface (5) and a bottom surface (6), ii. A first cell culture chamber (7) formed in the bottom surface (6) and in fluid communication with the through holes (4), iii. At least a first microfluidic channel (12) crossing the top surface (5) and in fluid communication with the first cell culture chamber (7), and iv. At least one sensor and/or an actuator (8) attached to the top surface (5) or the bottom surface (6), b. A second unit (9) comprising: i. A second cell culture chamber (10) formed above the top surface (5) of the first semiconductor substrate (3) and in fluid communication with the through holes (4), ii. A first optically transparent layer (11) covering the second cell culture chamber (10), and iii. An inlet (21) and an outlet (22) for allowing liquid to enter and exit the second cell culture chamber (10), and c. A second optically transparent layer (15) bonded with the bottom surface (6) of the first semiconductor substrate and covering the first cell culture chamber (7).

The semiconductor device (1) of the first aspect can be used as an organ-on-chips or a microphysiological system. A typical example of application for the semiconductor device is for modeling the blood/brain barrier. Other examples of applications are the modeling of the lung/blood barrier, the lung/air barrier, the vascular/blood barrier, and the gut/blood barrier. The semiconductor device (1) of the first aspect is a microfluidic device comprising semiconductor materials. It can be formed by using semiconductor technology. It can provide a chip-scale system for culturing cells in vitro. The semiconductor device (1) of the first aspect has the advantage of providing a three-dimensional platform for culturing cells in suspension. The semiconductor device (1) of the first aspect can be used for 3D cell culture applications. In particular, it is possible to have different three-dimensional (3D) cell cultures in the different cell culture chambers, i.e., different cells can grow in three dimensions within the different cell culture chambers.

The first semiconductor substrate (3) can be made of any semiconductor material. Preferably it is made of Silicon or Germanium. More preferably, it is made of silicon.

In embodiments, the first semiconductor substrate (3) may have a maximal thickness of less than 500 μm, such as from 35 to 450 μm.

In embodiments, the first semiconductor substrate (3) may be a single silicon wafer, preferably thinned down to less than 500 μm.

In embodiments, the first semiconductor substrate may be a monolithic structure.

The through holes (4) can be of any shape or size and can be formed by any suitable technique, such as etching or laser drilling. In embodiments, the through holes (4) in the first semiconductor substrate (3) have a maximum width measured parallel to the top surface (5) of the first semiconductor substrate (3) measuring from 3 μm to 200 μm, preferably from 5 μm to 150 μm, more preferably from 10 μm to 100 μm, or from 20 μm to 90 μm. The width of the through holes (4) affects the rate at which materials can be transported between cells in the first and second cell culture chambers (7, 20). In particular, the openings are preferably large enough so that the cells present at the top of the first cell culture chamber (7) can physically contact the cells present at the bottom of the second cell culture chamber (20). This is advantageous as this mimics the situation present in a human body, e.g., at the blood-brain barrier. Endothelial cells cannot bridge pores bigger than 100 μm and have difficulties bridging pores bigger than 90 μm.

A maximum through hole width measured parallel to the top surface (5) of the first semiconductor substrate (3) measuring from 20 μm to 90 μm is, therefore, particularly advantageous when the device is foreseen for growing endothelial cells on top of the top surface (5).

Although the first and the second cell culture chambers may have different widths, they typically have the same width. Although the first and the second cell culture chambers may have different lengths, they typically have the same length (the length is depicted as the depth in FIG. 1 and is perpendicular to the plane of the page). In embodiments, each of the first and the second cell culture chambers may have a different or a same width of from 0.5 to 3 mm. In embodiments, each of the first and the second cell culture chamber may have a same or a different height of up to 20 mm. More typically, the first cell culture chamber may have a height of from 100 μm to 500 μm, such as from 100 μm to 450 μm or from 100 μm to 420 μm. In embodiments, each of the first and the second cell culture chamber may have a same or a different length, preferably equal to or larger than its width. In embodiments, each of the first and the second cell culture chamber may have a same or a different length of up to 400 mm. Such dimensions of the first cell culture chamber (7) and the second cell culture chamber (10) are suitable to provide a good environment for the cell culture.

The first cell culture chamber (7) is formed in the bottom surface (6) by removing material, e.g., via etching the bottom surface of the semiconductor substrate. It is a chamber which sidewalls are at least partly delimited by the bottom surface (6) of the substrate. The height of the first cell culture chamber must be smaller than the maximal thickness of the first semiconductor substrate since it is formed by removing material from said substrate. For instance, the height of the first cell culture chamber may be from 100 μm to 500 μm, such as from 100 μm to 450 μm or from 100 μm to 420 μm.

The at least a first microfluidic channel (12) has an inlet (13) to the channel. This inlet (13) can, for instance, be an opening in the top surface (5) of the first semiconductor substrate (3) or a tube projecting from this top surface (5). An outlet (14) to the channel is also typically present. This outlet (14) can, for instance, be an opening in the top surface (5) of the first semiconductor substrate (3) or a tube projecting from this top surface (5).

The at least a first microfluidic channel (12) can be manufactured by etching, lithography, or any other suitable technique. The at least a first microfluidic channel (12) can be used for delivering nutrients (e.g., oxygen or glucose) or drugs to the first cell culture chamber (7), for removing waste from the first cell culture chamber (7), or for removing effluent of the cell culture for further processing. The width of the at least a first microfluidic channel (12) can, for instance, be from 0.2 to 1.5 mm.

In embodiments, the at least a first microfluidic channel (12) are at least two first microfluidic channels in fluid communication, e.g., in direct fluid communication, with opposite sides of the first cell culture chamber (7). The use of two microfluidic channels provides a better control over the environment of the cells in the first cell culture chamber (7). It also permits to form a chemical gradient between the opposite sides of the first cell culture chamber (7). This is advantageous as it allows to mimic gradients naturally occurring in the human body and/or allows to study a response of cells in presence of a gradient of a drug.

An example of a first microfluidic channel (12) is shown in some details in FIG. 2. In embodiments, the at least a first microfluidic channel (12) has an inlet (13) and an outlet (14) for fluids crossing the top surface (5) or being above the top surface (5) of the first semiconductor substrate (3), and a channel (12) fluidly connecting the inlet (13) and outlet (14) of the first microfluidic channel (12), said channel comprising a plurality of openings (17) in fluid communication, e.g., in direct fluid communication, with the first cell culture chamber (7), said openings (17) being adapted for letting fluid exit the channel while simultaneously preventing a gel, e.g. a hydrogel, from entering the channel. The width (d) of the openings (17) measured at the side of the channel (12) can be from 50 to 250 μm. In embodiments, the width (d) of the openings (17) measured at the side of the channel (12) can be smaller than the width (d′) of the openings (17) measured at the side of the first cell culture chamber (7). In embodiments, the openings can be formed by spaced pillars (27), wherein each pillar (27) bridges the bottom surface of the channel and the top surface of the channel (12). In embodiments, each pillar (27) may have a trapezoidal, triangular, square, or circular horizontal cross-section. In the case of a trapezoidal horizontal cross-section, a side parallel to a sidewall of the channel (12) and facing the inside of the channel (12) may be larger than a side facing the inside of the first cell culture chamber (7).

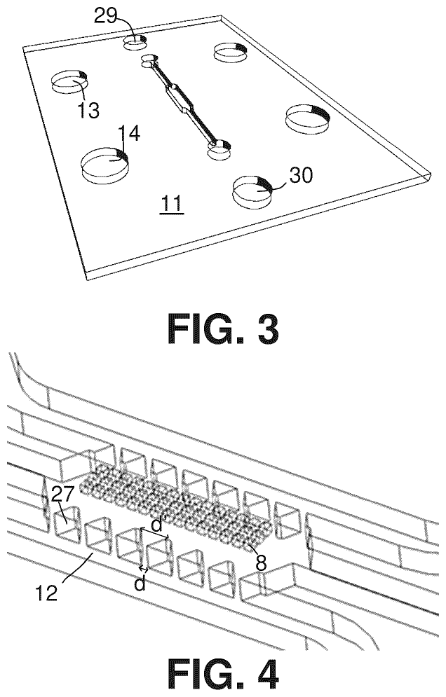

In embodiments, the device may further comprise a third microfluidic channel (28), separate from the first (see above) and second (see infra) microfluidic channels, for introducing the gel in the first, and if present in the third (see infra), cell culture chamber. This third microfluidic channel comprises its own inlet (29) and outlet (30). The channel is visible in FIG. 2 and the inlet (29) and outlet (30) are visible in FIGS. 2 and 3.

In embodiments, the semiconductor device (1) comprises further driving means for continuously driving a fluid from the inlet (13) to the outlet (14) of the at least a first microfluidic channel (12). The driving means can be a syringe pump or any other type of pump.

The at least one sensor and/or an actuator (8) can be any type of sensor or actuator (8) suitable for measuring or controlling parameters inside the first cell culture chamber (7). They are typically electrically connected to a printed circuit board (26). The printed circuit board (26) is adapted for sending electrical signals to the at least one sensor and/or actuator (8) and/or for receiving electrical signals from the at least one sensor and/or actuator (8). Aside from electrodes, these other sensing/actuator (8) elements can be any type of device that can be used to detect, measure, or influence a biological process, such as temperature sensors, humidity sensors, pH sensors, biological recognition elements, light emitters, light detectors, lasers, ultrasound transducers, and the like. The sensor and/or actuator (8) can be attached to the top surface (5) or the bottom surface (6) of the first semiconductor substrate (3). Preferably, they are attached to the top surface. One way of having the sensor and/or actuator (8) attached to the semiconductor substrate is by having the sensor and/or actuator (8) integrated to the semiconductor substrate by using the semiconductor substrate as a part of the sensor and/or actuator (8). Another way of having the sensor and/or actuator (8) attached to the semiconductor substrate is by manufacturing it separately from the semiconductor substrate, then bonding it with the semiconductor substrate.

Preferably, a plurality of sensors is present as it gives more reliable and sensitive data than a single sensor.

In embodiments, the at least one sensor and/or actuator (8) is an electrode array. The electrode array can be used for measuring the electrical properties of the cells, when they are present, such as the resistance or impedance between electrodes of the array.

In embodiments, the semiconductor device (1) comprises other electrodes than the electrodes of the array and the semiconductor device (1) is configured for measuring the resistance or impedance between at least one electrode of the electrode array and at least one of said further electrodes. This can be used for measuring the electrical properties of the cells, when they are present, e.g., by measuring the resistance or impedance, between different electrodes on the chip.

In embodiments, the sensor and/or actuator (8) on the top surface (5) or the bottom surface of the first substrate is adapted for performing an impedance spectroscopy. This can be used for measuring the electrical properties of the cells, such as the resistance or impedance, in the first cell culture chamber (7), when biological cells (23) are present.

In embodiments, the semiconductor substrate comprises an array of semiconductor islands connected by semiconductor bridges, wherein the through holes (4) occupy the space not occupied by the islands and the bridges, and wherein each of the at least one sensor and/or actuator (8) is attached to the top surface (5) or the bottom surface (6) of a semiconductor island. This is advantageous because the semiconductor substrate, which is typically a monolithic structure, can serve many important functions at once. It can simultaneously serve as a base for the formation of the second unit, as a communication port between the first and the second unit (via the through holes), as 5 of the 6 walls of the first cell culture chamber, and as support with dedicated locations for the at least one sensor and/or actuator (8) (via the semiconductor islands).

In embodiments, the first cell culture chamber (7) comprises a gel, such as a hydrogel. The hydrogel can provide a three-dimensional environment for the cells to grow in. The gel can be brought into the first chamber using the first microfluidic channel (12) crossing the top surface (5) as an access port to the first chamber. However, in preferred embodiments, the gel may be brought by the third microfluidic channel (28). Preferably, the gel is brought into the first chamber as a gel precursor liquid, before to be gelled. Gelling can be achieved by polymerizing the gel precursor liquid. The polymerization can be achieved by heat, light (e.g., UV light), a chemical reaction, or a combination thereof.

The second unit (9) is formed above the top surface (5) of the first semiconductor substrate (3). The second cell culture chamber (10) is formed by the second unit (9) and is in fluid communication with the through holes (4) of the first semiconductor substrate (3). The second cell culture chamber (10) can be made of a semiconductor wafer and a transparent material, or it can be made entirely of a transparent material. In either case, the chamber will be fitted with a transparent material on top which can for instance be glass, quartz, or a polymer. In the case of a semiconductor wafer, the chamber can be formed by using standard semiconductor fabrication techniques, such as etching, lithography or laser drilling. For a transparent material such as glass, the chamber may be formed by cutting and bonding the glass material to the top surface (5) of the first semiconductor substrate (3). The second cell culture chamber (10) is covered with the first optically transparent layer (11). An example of a first optically transparent layer (11) is shown in FIG. 3. The first optically transparent layer (11) used for closing the chamber can be made of any suitable transparent material such as glass, quartz, or a polymer. The first optically transparent layer (11) is formed by a material which allows for optical analysis of cells when they are present in the second cell culture chamber (10). The inlet (21) and outlet (22) of the second cell culture chamber (10) are typically crossing the top surface of the first optically transparent layer (11, see FIG. 3) and can be made by any suitable technique, such as etching, laser drilling, or lithography. The inlet (21) and outlet (22) of the second cell culture chamber (10) can be connected to a fluidic network which can be used for supplying nutrients and drugs to the second cell culture chamber (10).

The second optically transparent layer (15) is bonded with the bottom surface (6) of the first semiconductor substrate and covers the first cell culture chamber (7). This allows for optical analysis of the cells in the first cell culture chamber (7). The second optically transparent layer (15) may either be bound directly with the bottom surface (6) of the first semiconductor substrate or may be bound to it indirectly by being bound directly to the bottom surface of the second semiconductor substrate (see infra) or even to the bottom surface of a subsequent semiconductor substrate if more than three units are present. In embodiments, the second optically transparent layer (15) seals the bottom of the first cell culture chamber (7). The transparent material used for closing the chamber can be any suitable transparent material such as glass, quartz, or a polymer. The second optically transparent layer (15) is formed by a material which allows for optical analysis of cells when they are present in the first cell culture chamber (7).

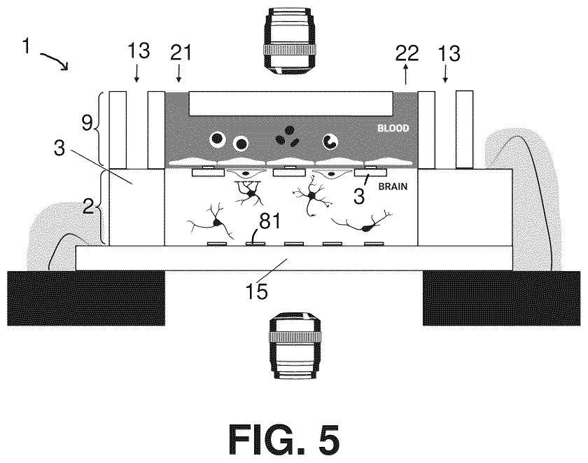

We now refer to FIG. 5. In embodiments, the second optically transparent layer (15) is not only a passive structural component but also serves as an active sensing platform. The second optically transparent layer (15) may have detectors and/or effectors (81) integrated onto its surface. They may be in electrical communication with each other. They may be in electrical communication with sensor and/or actuator (8) of the first unit (2). They may be in electrical communication with sensor and/or actuator (8) of one or more unit(s) between the first unit (2) and the second optically transparent layer (15), when present.

In embodiments, the detectors and/or effectors (81) on the second optically transparent layer (15) are patterned in such a way that they form an array of sensing areas interspersed with transparent regions. This patterning allows for optical analysis of the cell cultures in regions of the second optically transparent layer (15) that are free of detectors and/or effectors (81), thereby providing uninterrupted visual access while enabling localized sensing capabilities.

Preferably, the detectors and/or effectors (81) integrated onto the second optically transparent layer (15) are fabricated from transparent conductive materials, such as indium tin oxide (ITO), fluorine-doped tin oxide (FTO), or conductive polymers. This is advantageous as it maintains the transparency of the layer. These detectors and/or effectors (81) may include electrodes, transistors, or other electronic devices capable of detecting electrical, chemical, or biological signals from the cell cultures.

The presence of detectors (81) on the second optically transparent layer (15) permits to sense biological material that can also be observed. Biological material higher up in the device is less clearly visible. This allows a good correlation between what is sensed and what is observed.

The detector elements (81) on the second optically transparent layer (15) may be electrically connected to a printed circuit board (26). This printed circuit board is adapted for sending electrical signals to the detectors (or effectors) and/or for receiving electrical signals from the detectors (or effectors), thereby enabling the measurement of electrical properties such as impedance, capacitance, or potential within the cell culture chambers.

In embodiments, the semiconductor device (1) is configured to perform measurements by utilizing the detector elements (81) on the second optically transparent layer (15) in conjunction with electrodes or sensor elements (not depicted) on the first transparent layer (11). This configuration allows for direct measurement of electrical resistance across the cell barrier, providing valuable information about the integrity and permeability of the cell barrier within the device.

In embodiments, the semiconductor device (1) is configured to perform measurements by utilizing the detector elements (81) on the second optically transparent layer (15) in conjunction with electrodes or sensor elements (8) on the first semiconductor substrate (3) or the second semiconductor substrate (19). This configuration allows for the monitoring of the evolution and activity of brain-like structures over time, providing valuable insights into their functionality.

In embodiments, a layer of biological cells (23) is present at the bottom of the second cell culture chamber (10). The layer of cells at the bottom of the second cell culture chamber (10) can be used as a barrier between the first and the second cell culture chamber. This barrier can for instance allow oxygen, glucose, and amino acids to pass through. The barrier may, simultaneously, limit the passage of most other substances.

In embodiments, the layer of biological cells (23) is a monolayer.

In embodiments, the layer of biological cells (23) is an endothelial cell barrier. The use of an endothelial cell barrier has the advantage of providing a more physiological environment for the cells in the second cell culture chamber (10).

In embodiments, when a gel is present in the first chamber, the layer of biological cells (23), e.g., the endothelial cell barrier, may be physically supported by the gel. In embodiments, when a gel is present in the first chamber, the layer of biological cells (23), e.g., the endothelial cell barrier, may be physically supported by the gel and by the top of the first semiconductor substrate (3). The use of a gel as a support for the endothelial cell barrier has the advantage of providing a more physiological environment for the cells in the second cell culture chamber (10).

In embodiments, the gel present in the first cell culture chamber (7) is mixed with biological cells (23). The use of a gel mixed with cells has the advantage of providing a more physiological environment for the cells in the first cell culture chamber (7).

In embodiments, the semiconductor device (1) further comprises: d. a third unit (18) comprising: i. a second semiconductor substrate (19) having through holes (24), a top surface (25) bonded with the bottom surface (6) of the first semiconductor substrate, and a bottom surface, ii. A third cell culture chamber (20) formed in the bottom surface of the second semiconductor substrate (19) and in fluid communication with the through holes (4) of the second semiconductor substrate (19), iii. At least a second microfluidic channel crossing the top surface (25) of the second semiconductor substrate (19) and in fluid communication with the third cell culture chamber (20), iv. At least one sensor and/or an actuator (8) bonded to the top surface (25) or bottom surface (26) of the second semiconductor substrate (19), wherein the second optically transparent layer (15) is bonded with the bottom surface (6) of the first semiconductor substrate by the intermediate of the bottom surface of the second semiconductor substrate, thereby covering at once both the first cell culture chamber (7) and the third cell culture chamber (20).

The second semiconductor substrate (19) can be made of any semiconductor material. Preferably it is made of Silicon or Germanium. More preferably, it is made of silicon.

In embodiments, the second semiconductor substrate (19) may have a maximal thickness of less than 500 microns, such as from 35 μm to 450 μm.

In embodiments, the second semiconductor substrate (19) may be a single silicon wafer.

In embodiments, the second semiconductor substrate (19) may be a monolithic structure.

The through holes (24) in the second semiconductor substrate (19) can be of any shape or size and can be formed by any suitable technique, such as etching or laser drilling. In embodiments, the through holes in the second semiconductor substrate (19) have a maximum width measured parallel to the top surface of the second semiconductor substrate (19) measuring from 3 μm to 200 μm, preferably from 5 μm to 150 μm, more preferably from 10 μm to 100 μm, or from 20 μm to 90 μm. The width of the through holes affects the rate at which nutrients and other materials can be exchanged between cells in the first and third cell culture chambers (7).

The shape of the though holes is not critical.

Although the first and the third cell culture chambers may have different widths, they typically have the same width. Although the first and the third cell culture chambers may have different lengths, they typically have the same length (the length is depicted as the depth in FIG. 1 and is perpendicular to the plane of the page). In embodiments, each of the first and the third cell culture chambers may have a different or a same width of from 0.5 to 3 mm. In embodiments, each of the first and the third cell culture chamber may have a same or a different height of up to 20 mm, e.g., from 100 μm to 500 μm such as from 100 μm to 450 μm or from 100 μm to 420 μm. In embodiments, each of the first and the third cell culture chamber may have a same or a different length, preferably equal to or larger than its width. In embodiments, each of the first and the third cell culture chamber may have a same or a different length of up to 400 mm. Such dimensions of the first cell culture chamber (7) and the third cell culture chamber (20) are suitable to provide a good environment for the cell culture.

The dimensions of the third cell culture chamber (20) are adapted to provide a good environment for the cell culture.

The third cell culture chamber (20) is formed in the bottom surface of the second semiconductor substrate (19) by removing material from, e.g., by etching the second semiconductor substrate. It is a chamber which sidewalls and top surface are at least partly delimited by the bottom surface of the second semiconductor substrate (19).

The height of the third cell culture chamber must be smaller than the maximal thickness of the third semiconductor substrate since it is formed by removing material from said substrate. For instance, the height of the third cell culture chamber may be from 100 μm to 500 μm such as from 100 μm to 450 μm or from 100 μm to 420 μm.

In embodiments, each of the at least a second microfluidic channel can be entirely separated from each of the at least a first microfluidic channel.

In other embodiments, each of the at least a second microfluidic channel can be in fluidic communication with each of the at least a first microfluidic channel (12).

The at least a second microfluidic channel can be manufactured by etching, lithography, or any other suitable technique. The at least a second microfluidic channel can be used for delivering nutrients or drugs to the third cell culture chamber (7), for removing waste from the third cell culture chamber (7), or for collecting effluent from the third cell culture chamber (7) for further processing. The width of the at least a second microfluidic channel can, for instance, be from 0.2 to 1.5 mm.

In embodiments, the at least a second microfluidic channel are at least two second microfluidic channels in fluid communication, e.g., in direct fluid communication, with opposite sides of the third cell culture chamber (10). The use of two microfluidic channels provides a better control over the environment of the cells in the third cell culture chamber (10). It also permits to form a chemical gradient between the opposite sides of the third cell culture chamber (10). This is advantageous as it allows to mimic gradients naturally occurring in the human body and/or allows to study a response of cells in presence of a gradient of a drug.

In embodiments, the at least a second microfluidic channel (12′) has an inlet (13′) and an outlet for fluids crossing the top surface (25) or being above the top surface (25) of the second semiconductor substrate (19), and a channel (12′) fluidly connecting the inlet (13′) and outlet (14′) of the second microfluidic channel, said channel comprising a plurality of openings (not depicted) in fluid communication with the third cell culture chamber (20), said openings being adapted for letting fluid enter the third cell culture chamber (20) while simultaneously preventing a gel, e.g., a hydrogel, from exiting the third cell culture chamber (20). The width of the openings measured at the side of the channel (12′) can be from 50 to 250 μm. In embodiments, the width (d) of the openings (17) measured at the side of the channel (12′) can be smaller than the width (d′) of the openings (17) measured at the side of the third cell culture chamber (7).

In embodiments, the openings can be formed by spaced pillars (27), wherein each pillar (27) bridges the bottom surface of the channel (12′) and the top surface of the channel (12′). In embodiments, each pillar (27) may have a trapezoidal, triangular, square, or circular horizontal cross-section. In the case of a trapezoidal horizontal cross-section, a side parallel to a sidewall of the channel (12′) and facing the inside of the channel (12′) may be larger than a side facing the inside of the third cell culture chamber (20).

In embodiments, when the semiconductor device (1) comprises further driving means for continuously driving a fluid from the inlet (13) to the outlet (14) of the at least a first microfluidic channel (12), these driving means may also be used to drive a fluid from the inlet (13′) to the outlet (14′) of the at least a second microfluidic channel (12′).

The at least one sensor and/or an actuator (8) can be any type of sensor or actuator (8) suitable for measuring or controlling parameters inside the third cell culture chamber (20). Preferably, said at least one sensor and/or actuator is at least capable of acting as a sensor, i.e., is capable of recording electrical activity from biological cells. Aside from electrodes, these other sensing/actuator (8) elements can be any type of device that can be used to detect, measure, or influence a biological process, such as temperature sensors, humidity sensors, pH sensors, biological recognition elements, light emitters, light detectors, lasers, ultrasound transducers, and the like. The sensor and/or actuator (8) can be attached to the top surface (25) or the bottom surface (26) of the second semiconductor substrate (19). One way of having the sensor and/or actuator (8) attached to the semiconductor substrate is by having the sensor and/or actuator (8) integrated to the semiconductor substrate by using the semiconductor substrate as a part of the sensor and/or actuator (8). Another way of having the sensor and/or actuator (8) attached to the semiconductor substrate is by manufacturing it separately from the semiconductor substrate, then bonding it with the semiconductor substrate.

In embodiments, the at least one sensor and/or actuator (8) is an electrode array. The electrode array can be used for measuring the electrical properties of the cells, when they are present, e.g., by measuring the resistance or impedance between electrodes of the array.

In embodiments, when the semiconductor device (1) comprises a first and a third unit (18), the semiconductor device (1) may be configured for measuring the resistance or impedance between at least one electrode of the electrode array of the first unit (2) and at least one electrode of the electrode array of the third unit (18). This can be used for measuring the electrical properties of the cells, when they are present, e.g., via measuring the resistance or impedance, between different units of the device.

In embodiments, the sensor and/or actuator (8) on the top surface (25) or bottom surface (26) of the second substrate (19) is adapted for performing an impedance spectroscopy. This can be used for measuring the electrical properties of the cells, such as the resistance or impedance, in the first cell culture chamber (7), when biological cells (23) are present.

In embodiments, the second semiconductor substrate (19) comprises an array of semiconductor islands connected by semiconductor bridges, wherein the through holes occupy the space not occupied by the islands and the bridges, and wherein each of the at least one sensor and/or actuator (8) is attached to the top surface (25) or the bottom surface (26) of a semiconductor island.

In embodiments, the third cell culture chamber (20) comprises a gel, such as a hydrogel. The hydrogel can provide a three-dimensional environment for the cells to grow in. The hydrogel can be brought into the third chamber (20) using the second microfluidic channel crossing the top surface as an access port to the third chamber. Alternatively, a separate microfluidic channel can be used as an access port for the gel to the third chamber.

In embodiments, the second optically transparent layer (15) seals the bottom of the third cell culture chamber (20). The second optically transparent layer (15) may have detectors and/or effectors (81) integrated onto its surface.

In the second aspect, the present invention relates to a process for fabricating a semiconductor device (1) comprising: a. providing a first semiconductor substrate (3) having through holes (4), a top surface (5) and a bottom surface (6); b. forming a first cell culture chamber (7) in the bottom surface (6) and in fluid communication with the through holes (4); c. forming at least a first microfluidic channel (12) crossing the top surface (5) and in fluid communication with the first cell culture chamber (7); d. forming at least one sensor and/or an actuator (8) on the top surface (5) or bottom surface (6); e. forming a second cell culture chamber (10) above the top surface (5) of the first semiconductor substrate (3) such that the first cell culture chamber (7) is in fluid communication with the second cell culture chamber (10); f. providing a first optically transparent layer (11); g. bonding the first optically transparent layer (11) to the top surface of the second cell culture chamber (10) so that it is covered by the first optically transparent layer (11); h. providing a second optically transparent layer (15); i. bonding the second optically transparent layer (15) to the bottom surface (6) of the first semiconductor substrate (3) such that the first cell culture chamber (7) is covered by the second optically transparent layer (15).

Any feature of the second aspect can be as correspondingly described for the first aspect.

It is to be understood that although preferred embodiments, specific constructions and configurations, as well as materials, have been discussed herein for devices according to the present invention, various changes or modifications in form and detail may be made without departing from the scope of this invention. For example, any formulas given above are merely representative of procedures that may be used. Functionality may be added or deleted from the block diagrams and operations may be interchanged among functional blocks. Steps may be added or deleted to methods described within the scope of the present invention.

1. A semiconductor device comprising:
a first unit comprising:
a first semiconductor substrate having through holes, a top surface and a bottom surface,
a first cell culture chamber formed in the bottom surface and in fluid communication with the through holes,
at least a first microfluidic channel crossing the top surface and in fluid communication with the first cell culture chamber, and
at least one sensor and/or an actuator attached to the top surface or the bottom surface; and
a second unit comprising:
a second cell culture chamber formed above the top surface of the first semiconductor substrate and in fluid communication with the through holes,
a first optically transparent layer covering the second cell culture chamber, and
an inlet and an outlet for allowing liquid to enter and exit the second cell culture chamber, and
a second optically transparent layer bonded with the bottom surface of the first semiconductor substrate and covering the first cell culture chamber.

2. The semiconductor device according to claim 1, wherein the at least one sensor and/or actuator is an electrode array.

3. (canceled)

4. The semiconductor device according to claim 2, wherein the semiconductor device comprises further electrodes and wherein the semiconductor device is configured for measuring the resistance or impedance between at least one electrode of the electrode array and at least one of the further electrodes.

5. The semiconductor device according to claim 1, wherein the first semiconductor substrate comprises an array of semiconductor islands connected by semiconductor bridges, wherein the through holes occupy the space not occupied by the islands and the bridges, and wherein each of the at least one sensor and/or actuator is attached to the top surface or the bottom surface of a semiconductor island.

6. The semiconductor device according to claim 1, wherein the first cell culture chamber comprises a gel.

7. The semiconductor device according to claim 1, wherein the sensor and/or actuator on the top surface of the first semiconductor substrate is adapted for performing an impedance spectroscopy.

8. (canceled)

9. The semiconductor device according to claim 1, wherein the at least first microfluidic channel has an inlet and an outlet for fluids crossing the top surface or being above the top surface of the first semiconductor substrate, and a channel fluidly connecting the inlet and outlet of the first microfluidic channel, the channel comprising a plurality of openings in fluid communication with the first cell culture chamber, the openings being adapted for letting fluid exit the channel while simultaneously preventing a gel from entering the channel.

10. The semiconductor device according to claim 9, wherein the openings in fluid communication with the first cell culture chamber have a width of from 50 to 250 μm.

11. (canceled)

12. The semiconductor device according to claim 1, wherein the at least a first microfluidic channel are at least two first microfluidic channels in fluid communication with opposite sides of the first cell culture chamber.

13. The semiconductor device according to claim 1, wherein a layer of biological cells is present at the bottom of the second cell culture chamber.

14. The semiconductor device according to claim 13, wherein the layer of biological cells is a monolayer, and, optionally, wherein the layer of biological cells is an endothelial cell barrier.

15. (canceled)

16. (canceled)

17. The semiconductor device according to claim 6, wherein the gel present in the first cell culture chamber is mixed with biological cells.

18. The semiconductor device according to claim 1, further comprising:
a third unit comprising:
a second semiconductor substrate having through holes, a top surface bonded with the bottom surface of the first semiconductor substrate, and a bottom surface,
a third cell culture chamber formed in the bottom surface of the second semiconductor substrate and in fluid communication with the through holes of the second semiconductor substrate,
at least a second microfluidic channel crossing the top surface of the second semiconductor substrate and in fluid communication with the second cell culture chamber and with one of the at least a first microfluidic channel, and
at least one sensor and/or an actuator bonded to the top surface of the second semiconductor substrate,
wherein the second optically transparent layer is bonded with the bottom surface of the first semiconductor substrate by the intermediate of the bottom surface of the second semiconductor substrate, thereby covering at once both the first cell culture chamber and the third cell culture chamber.

19. (canceled)

20. The semiconductor device according to claim 1, wherein the second optically transparent layer comprises integrated detector and/or effector elements on its surface, the detector and/or effector elements being in electrical communication with each other.

21. The semiconductor device according to claim 20, wherein the detector and/or effector elements integrated onto the second optically transparent layer are fabricated from transparent conductive materials.

22. The semiconductor device according to claim 21, wherein the transparent conductive materials are selected from the group consisting of indium tin oxide (ITO), fluorine-doped tin oxide (FTO), and conductive polymers.

23. The semiconductor device according to claim 20, wherein the detector and/or effector elements on the second optically transparent layer are patterned to form an array of sensing areas interspersed with transparent regions.

24. The semiconductor device according to claim 20, wherein the detector and/or effector elements on the second optically transparent layer are electrically connected to a printed circuit board.

25. The semiconductor device according to claim 20, wherein the semiconductor device is configured to perform Transepithelial Electrical Resistance (TEER) measurements using the detector elements on the second optically transparent layer in conjunction with sensor elements on the first semiconductor substrate or the second semiconductor substrate.

26. (canceled)

27. (canceled)

28. A process for fabricating a semiconductor device comprising:
providing a first semiconductor substrate having through holes, a top surface and a bottom surface;
forming a first cell culture chamber in the bottom surface and in fluid communication with the through holes;
forming at least a first microfluidic channel crossing the top surface and in fluid communication with the first cell culture chamber;
forming at least one sensor and/or an actuator on the top surface or bottom surface;
forming a second cell culture chamber above the top surface of the first semiconductor substrate such that the first cell culture chamber is in fluid communication with the second cell culture chamber;
providing a first optically transparent layer;
bonding the first optically transparent layer to the top surface of the second cell culture chamber so that it is covered by the first optically transparent layer;
providing a second optically transparent layer; and
bonding the second optically transparent layer to the bottom surface of the first semiconductor substrate such that the first cell culture chamber is covered by the second optically transparent layer.
